(19) United States
(12) Patent Application Publication
(10) Pub. No.: US 2013/0330402 A1
(43) Pub. Date: Dec. 12, 2013

(54) ANTINUCLEAR ANTIBODY UTILIZED AS A TARGETING AGENT FOR PHARMACEUTICAL COMPOUNDS USED IN THE TREATMENT OF CANCER AND OTHER DISEASES

(71) Applicants: HENRY J. SMITH, TEMECULA, CA (US); JAMES R. SMITH, LAGUNA NIGUEL, CA (US)

(72) Inventors: HENRY J. SMITH, TEMECULA, CA (US); JAMES R. SMITH, LAGUNA NIGUEL, CA (US)

(21) Appl. No.: 13/970,099

(22) Filed: Aug. 19, 2013

Related U.S. Application Data
(63) Continuation of application No. 12/584,159, filed on Sep. 2, 2009.

(51) Int. Cl. A61K 47/42

(52) U.S. Cl. CPC A61K 47/42
USPC 424/450; 424/130.1; 424/174.1; 424/491

(57) ABSTRACT

This invention describes a method whereby autoimmune antinuclear antibodies are used as a targeting agent to deliver drug nanoparticles or drug liposomes to the tumor or disease site. The antinuclear antibodies have the propensity to localize in areas of tissue necrosis where dead cells have released their nuclear material into the extracellular environment. Many tumors have areas of necrosis that can be targeted using antinuclear antibody coated drug nanoparticles or liposomes. Similarly, many infectious diseases have areas of necrosis and can also be targeted using antinuclear antibody coated drug nanoparticles or liposomes. Similarly, many immune disorders such as rheumatoid arthritis and osteoarthritis have areas of inflammation where there is cell death, and these inflammatory sites can also be targeted using antinuclear antibody coated drug nanoparticles or liposomes.

### CROSS-REFERENCE TO RELATED APPLICATIONS

This application claims priority to provisional patent application No. 61/195,237 entitled ‘Antinuclear antibody utilized as a targeting agent for pharmaceutical compounds used in the treatment of cancer and other diseases’ filed Oct. 6, 2008.

### STATEMENT REGARDING FEDERALLY SPONSORED RESEARCH OR DEVELOPMENT Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

There is intensive ongoing research into developing improved methods for treating cancer and other diseases. One out of every four people in the US will die from cancer; two million people in the US have rheumatoid arthritis; and there are thousands of patients hospitalized each year with infection. There is a great need to develop new pharmaceuticals and new delivery systems for treating these diseases:

Almost all the research into immunotherapy for cancer has focused on developing antitumor antibodies that have selective binding capacity for tumor associated antigens. Early research on targeting tumors used antibodies obtained from immunized animals. Subsequent studies have been almost exclusively devoted to developing “humanized” monoclonal antibodies against tumor associated antigens or to over-expressed normal cell markers. For example, Herceptin^{R} is a monoclonal antibody that targets the overexpressed cell marker HER2 on the surface of breast cancer cells; Erbitux^{R} is a monoclonal antibody that targets epidermal growth factor receptor on colon cancer cells; and Zevalin^{R} is monoclonal antibody labeled with a radioisotope that can inhibit non-Hodgkin's lymphoma cells.

Treatment for infectious disease is typically focused on developing new antibiotics that are effective against particular species or strains of bacteria and microbes. There is limited research into developing .delivery systems to ensure that most of the administered antibiotic reaches the site of infection.

Current treatment for immune diseases and inflammatory disorders such as rheumatoid arthritis and osteoarthritis typically consists of administering non-steroidal drugs for mild cases, and using steroidal drugs such as cortisone or cytotoxic agents such as methotrexate for more severe disease. More recently, immune modulating agents such as Enbrel^{R} a recombinant anti-TNF alpha receptor and Remicade^{R} a monoclonal antibody that inhibits tumor necrosis factor have been developed.

Conventional wisdom would suggest that in order to treat a disease one must identify the pathogen or pathogenic factors causing the disease; and then devise a drug and/or a treatment method that would cause the drug to directly act upon the pathogen or pathogenic factor causing that disease.

We have made the surprising discovery that there is an alternative method of treating disease based on a fundamentally different concept from that described above. Our invention describes an unconventional targeting system for selectively delivering pharmaceuticals to the disease site. The novelty of our invention is that our targeting system does not specifically target the pathogen or pathogenic factors characteristic for a particular disease, but instead targets areas of necrosis caused by the disease. There are many diseases such as cancer, and/or infection, and/or inflammation where the disease causes cell death resulting in areas of necrosis. Our targeting moiety is an antibody that targets certain antigens present in necrotic areas and therefore can be used to treat any disease that has accompanying necrosis.

The delivery system of this invention consists of two components. A nanoparticle or liposomal formulation of a drug; and a “necrosis” targeting antibody attached to the surface of the nanoparticle or liposome. A further novelty of this invention is that autoimmune antinuclear antibodies are used as the “necrosis” targeting moiety for delivering a wide variety of different pharmaceuticals to the disease site of very different diseases such as cancer, infectious disease and immune disorders.

It is well known that many diseases result in some degree of tissue destruction. For example, many tumors have areas of necrosis and these necrotic areas contain elevated levels of intracellular material released from dead or dying cells. Similarly, many diseases such as infectious diseases also have areas of necrosis within the infection site. Similarly, immune diseases and inflammatory disorders may also have areas of necrosis within the affected joint or tissue inflammation site. The extracellular expressed material includes nuclear components such as the nuclear membrane, nucleoproteins, double-stranded DNA, single-stranded DNA, extractable nuclear antigen (ENA), ribonucleoprotein (RNP), Sm antigen and other nuclear antigens.

Autoimmune antinuclear antibodies (ANA) are commonly present in the blood of patients with autoimmune disease. High titers of antinuclear antibodies are characteristic of patients with active systemic lupus erythematosus (SLE). Immunological testing in vitro using the indirect fluorescent assay (IFA) and the enzymelinked-immunosorbent assay (ELISA) have shown that antinuclear antibodies will bind to a variety of nuclear antigens such as deoxyribonucleoprotein (DNP); double-stranded DNA, single-stranded DNA, extractable nuclear antigen (ENA), ribonucleoprotein (RNP), Sm antigen and other nuclear antigens.

Administration of antinuclear antibody into patients who have a diseased site with areas of tissue necrosis will result in the ANA binding to the extracellular nuclear material present in necrotic areas. In this regard the antibody of this invention can be characterized as a “necrosis-specific” antibody. Therefore attaching the ANA to a drug nanoparticle or liposome will cause them to concentrate within necrotic areas where the drug can be released to act upon the surrounding pathogenic elements whether they are cancer cells, or bacterial cells, or inflammatory cells.

Healthy tissues do not have areas of necrosis and therefore the ANA conjugated drug nanoparticles or liposomes cannot bind to and affect healthy tissues.

A further benefit of this invention is that because the antinuclear antibody is obtained from human donors it will not provoke an allergic reaction when administered to the patient.

### SUMMARY OF THE INVENTION

This invention describes the novel use of antinuclear antibodies as carrier agents for pharmaceutical compounds used to treat cancer and other diseases. The invention describes the utilization of antinuclear antibodies that have the capacity of binding to intracellular components released from dead or dying cells. Further, this invention describes the process whereby these antinuclear antibodies are used to coat drug nanoparticles and/or drug liposomes in order to cause the drug to localize within the necrotic areas found in many tumors, and in infectious diseases, and in inflammatory sites.

There are two components to this invention. First, there is the preparation and composition of drug nanoparticles or drug liposomes to impart certain properties to the drug that will enhance its efficacy and safety. Second, is to “coat” the drug nanoparticle or drug liposome with the antinuclear antibodies described herein in order to further improve the safety and efficacy profile of the drug by causing it to selectively localize at the disease site where it will have optimum effect.

The antinuclear antibodies being of themselves non-immunogenic can be used repeatedly as “carriers” for pharmaceuticals without provoking an adverse allergic reaction in the patient.

### DESCRIPTION OF THE INVENTION

This invention describes a methodology for improved delivery of pharmaceutical agents to treat a variety of tumors and other diseases using specific antinuclear antibodies combined with various pharmaceutical compounds formulated as drug nanoparticles and/or liposomes. Although the term nanoparticle can be applied to any material that is measured in nanometers it is generally applied to particles that are between 1 nanometer and 1000 nanometers in size. Nanoparticles can be broadly recognized as two types; those particles that have a solid structure and those that have a liquid structure. For purposes of clarity in this invention the term “nanoparticle” will refer to the solid type of particle, and because of convention the term “liposome” will be used to describe the liquid type. In general, if the drug is insoluble or poorly soluble in physiological solution it is made into nanoparticles or incorporated into the lipid membrane of liposomes. If the drug is soluble in physiological solution it is typically encapsulated into liposomes. In certain instances the liquid drug may be incorporated in a solid or gel-matrix which is then made into nanoparticles.

The drug nanoparticle and/or the drug liposome can be treated so that the surface layer will incorporate additional chemical or biological agents. This invention describes the advantages of attaching a unique antinuclear antibody to the surface of the nanoparticle or liposome to further improve its therapeutic profile. It will also cause the drug nanoparticles or drug liposomes to accumulate within necrotic areas by binding to intracellular antigens released from dead cells. We refer to the drug delivery system described herein as a “smart” delivery system because it consists of two components: a targeting component which is the carrier antinuclear antibody, and the payload component which is the drug nanoparticle and/or drug liposome that is delivered to the disease site. Hence the terms “smart nanoparticles” and “smart liposomes”.

In a further embodiment of this invention the carrier antinuclear antibody is attached to the drug nanoparticle or drug liposome through a polyethylene glycol (PEG) linkage molecule. The PEG coating molecule helps to protect the nanoparticle or liposome from being broken down by the liver and therefore results in more of the drug being bioavailable. This is often referred to as a “stealthy” nanoparticle or liposome.

This invention is based on the observation that certain antinuclear antibodies obtained from patients with SLE have a unique propensity for binding to certain intracellular material found extracellularly within necrotic areas of certain diseases but not in healthy tissues. The invention describes the process whereby various therapeutic agents in the form of nanoparticles and/or liposomes are combined with these antinuclear antibodies and used in the treatment of cancer and other diseases.

A major benefit of this invention is that these antinuclear antibodies being human derived are non-immunogenic, and therefore patients can receive repeated treatment without eliciting an adverse allergic response.

There are a variety of antinuclear antibodies that can be employed as targeting carriers for pharmaceuticals. These include antinuclear antibodies directed against the intracellular nuclear components of the cell such as deoxyribonucleoprotein (DNP), double-stranded DNA (dsDNA), single-stranded DNA (ssDNA), Extractable Ribonucleoprotein Antigen (ENA), ribonucleoprotein (RNP), Sm antigen, and other nuclear antigens.

Antibody Preparation.

Antinuclear antibodies are commonly found in patients with SLE or other autoimmune diseases. Blood from the SLE patient is collected and allowed to clot. The serum containing the autoantibody of interest is fractionated by standard laboratory techniques in order to concentrate and purify the autoantibodies. These procedures are known to those skilled in the art. For example, one purification process is to concentrate the immunoglobulin fraction using ammonium salt precipitation and gel-filtration; followed by affinity chromatography to isolate the desired autoantibodies. For example, the immunoglobulin fraction in serum is precipitated by adding saturated ammonium sulphate solution to reach a final concentration of 33% saturation; the precipitated immunoglobulin fraction is dissolved in phosphate buffer solution and dialyzed against the buffer to remove any remaining ammonium sulphate. The antinuclear antibodies in the immunoglobulin fraction are then isolated using affinity chromatography or affinity binding techniques.

Blood or blood products from individuals with autoimmune disease are available from blood donation or from commercial sources. There are various procedures for blood donation. Either whole blood is donated, or a procedure termed “plasmapheresis” is used in which a specified volume of the donor's blood is processed through a cell separator machine that removes the plasma and returns the blood cells to the patient. There is also a process termed “apheresis” in which the donor's blood is continuously processed through a machine that removes the immunoglobin fraction and returns the other components of the blood to the patient. The immunoglobulin fraction includes the pathogenic antinuclear autoantibodies that are involved in the disease process, and therefore their removal may ameliorate the symptoms of the disease. Apheresis has been used to treat a number of autoimmune diseases including systemic lupus erythematosus (SLE).

In one embodiment of the invention the patient with SLE is connected to the apheresis machine and the blood is circulated over an affinity chromatography column. The affinity column is typically composed of either Protein A or anti-human immunoglobin antibody that is bound to an insoluble support such as agarose or sepharose contained in a cartridge. The affinity column binds out the immunoglobulin fraction of the blood allowing the other blood components to return to the patient. It is likely that in the future affinity columns will be developed that will bind out only the pathogenic autoantibodies and allow the remaining immunoglobulins and other components of the blood to be returned to the patient.

The immunoglobulin fraction of the blood obtained from SLE patients is purified using affinity binding to obtain a highly purified preparation of antinuclear antibodies.

In one embodiment of this invention fixed whole isolated nuclei are used as the affinity binding ligand. The nuclei can be obtained from many different human and animal sources. In the preferred embodiment of this invention nuclei obtained from human cells are used. For example, tissue culture human cell lines such as Hela or Hep 2 can be used; or buffy coat cells obtained from human blood. The cells are suspended in an osmotically balanced sucrose solution and disrupted using a mechanical homogenizer. Intact cells and large particulate debris are sedimented using low speed centrifugation and discarded. The supernatant is then centrifuged at a higher speed to sediment the isolated nuclei. The isolated nuclei are resuspended in a low volume of buffer and fixed by adding a fixative such as ethyl alcohol or glutaldehyde. After fixation the nuclei are washed several times in phosphate buffer to remove any remaining fixative and suspended in phosphate buffered saline (PBS). The immunoglobulin fraction of SLE sera is mixed with the nuclei for several hours at room temperature with gentle agitation to allow antibody binding to occur. The mixture is then centrifuged to sediment the nuclei and the supernatant removed. The isolated nuclei with bound antinuclear antibody are then washed several times with PBS and the bound antibody is then eluted off the nuclei using a low pH glycine-HCl buffer. The eluted antibody solution is neutralized using a NaOH solution and diluted in phosphate buffer pH 7.2. The purified antinuclear antibody preparation is tested for its protein concentration and its ANA activity measured using an enzymelinked-immunoassay (ELISA) for ANA. The results indicate a highly purified antibody preparation was achieved using this procedure. It is obvious that there are many other purification methods known to those skilled in the art that may be employed without affecting the novelty of this invention.

In one embodiment of this invention a fixed nuclear extract immobilized on an insoluble matrix and made into an affinity column is used. The nuclear extract can be prepared from isolated nuclei obtained from human or animal cells but human cells are the preferred source. The isolated nuclei fraction is prepared as described earlier, but this time the isolated nuclei are disrupted using a mechanical homogenizer or repeated freeze thaw cycles. The nuclear extract is centrifuged to sediment large particulate material and the supematant containing small particles and soluble nuclear material is used to prepare an affinity column. The nuclear extract is fixed to an insoluble support such as cross-linked agarose beads or sepharose^{R} beads. The agarose beads are activated using cyanogen bromide and the activated beads are incubated with the nuclear extract to chemically fix them to the beads. After washing with PBS to remove uncoupled material the ligand bound beads are used to prepare an affinity column. The immunoglobulin fraction of SLE sera is applied to the column to allow antibody binding to the immobilized ligand to occur. After washing the column with PBS the bound antibody is eluted off the column using a low pH glycine-HCl buffer. The eluate fractions are adjusted to neutral pH and tested for antinuclear antibody using an ELISA test for ANA. The results indicate a highly purified antibody preparation was achieved using this procedure. It is obvious that there are many other purification methods known to those skilled in the art that may be employed without affecting the novelty of this invention.

Other methods of obtaining large quantities of human antinuclear antibodies include using fully human hybridomas in which antibody producing autoimmune cells from patients with autoimmune disease such as SLE are fused with a human cell line to produce monoclonal autoimmune antibodies. Another method utilizes transgenic animals in which the animal's immune system is replaced with a human immune system. By transferring immune cells from a patient with autoimmune disease into the transgenic animal it can be induced to produce fully human autoantibodies. In all these instances the prime source material for obtaining antinuclear antibodies are antibody cells obtained from SLE patients; and therefore these and other methods that utilize SLE patients as source material are considered to be within the scope of this invention.

In certain situations it may be preferable to use the binding fragments Fab or F(ab′)₂ of the antibody molecule as the carrier protein and this also falls within the scope of this invention. In this context, the terms “autoimmune antibodies” and “autoantibodies” refer to either the whole intact IgG or IgM antibody molecule or to the binding fragments F(ab′)₂ and Fab of the antibody molecule.

The purified antinuclear antibody can be combined with a wide variety of pharmaceutical compounds and used to treat cancer and other diseases.

In one embodiment of this invention the antinuclear antibody is attached to the surface of a drug nanoparticle and used to selectively transport the drug to necrotic areas found within tumors or tissues damaged by infection or inflammatory diseases.

In another embodiment of this invention the antinuclear antibody is attached to the surface of a drug liposome and used to selectively transport the drug to necrotic areas found within tumors or tissues damaged by infection or inflammatory diseases.

There are many methods of preparing nanoparticles known to those skilled in the art. These include dry and wet milling; super critical fluid technology, spray drying, solvent precipitation and recrystallization techniques. These methods are extensively modified to suit the requirements of the particular drug being used. These methods are known to those skilled in the art and are within the scope of this invention.

Similarly, there are many methods of preparing liposomes known to those skilled in the art. These include encapsulation, partitioning, and reverse loading techniques. As before, these methods are extensively modified to suit the requirements of the particular drug being used. These methods are known to those skilled in the art and are within the scope of this invention.

There are a large variety of pharmaceutical compounds used for the treatment of various cancers, infectious diseases and inflammatory conditions. Many of these can be combined with antinuclear antibodies and made into “smart nanoparticles” and/or “smart liposomes”. The methods of attaching antinuclear antibodies to the surface of drug nanoparticles or drug liposomes are known to those skilled in the art and are considered to be within the scope of this invention.

In one embodiment of this invention the antinuclear antibody is linked to a polyethylene glycol (PEG) chain which is in turn attached to the surface of the drug nanoparticle or drug liposome. The new pharmaceutical formulation is described as “smart” and “stealthy” in that it can selectively deliver the drug to the disease site and also avoid being destroyed by the liver and the reticuloendothelial system.

Example of a “smart and stealthy” liposome.

The liposomes can be prepared using a mixture of one or more of the following compounds: egg phosphatidylcholine (EPC), hydrogenated soy phosphatidylcholine (HSPC), phosphatidylethanolamine (PE), phosphatidylglycerol (PG), phosphatidylinsitol (PI), monosialoganglioside and sphingomyelin (SPM); the derivatized vesicle forming lipids such as poly(ethylene glycol)-derivatized distearoylphosphatidylethanolamine (DSPE-PEG), poly(ethylene glycol)-derivatized ceramides (CER-PEG), distearoylphosphatidylcholine (DSPC), dimyristoyl-phosphatidylcholine (DMPC), dimyristoylphosphatidylglycerol (DMPG), and dipalmitoylphosphatidylcholine (DPPC), and cholesterol. A stabilizing protein is preferably used to generate stabilized liposomes. In one embodiment, to generate stabilized liposomes, human serum albumin is added to act as a stabilizing agent. Other stabilizing agents that may be used in lieu of albumin include gelatin, casein and whey protein.

In this invention a quantity of 1,2-Distearoyl-sn-Glycero-3-Phosphoethanolamine-N-PDP [PolyethyleneGlycol 2000] (DSPE-PEG) and 1,2-Distearoyl-sn-Glycero-3phosphoethanolamine-N-Maleimide[Polyethylene Glycol 2000] (DSPE-PEG Maleimide) is added to the lipid mixture. Liposomes that have DSPE-PEG incorporated into the membrane bilayer can avoid recognition and destruction by liver cells and will have a longer residence time within the blood circulatory system. These liposomes are often referred to as “stealthy” liposomes. Also anchored to the liposome membrane is DSPE-PEG maleimide, with the maleimide group being free and able to be chemically coupled to the Fab fragment of the antinuclear antibody through an amide bond. Liposomes that have a targeting moiety attached are often referred to as “smart” liposomes.

The following example illustrates the preparation of a “smart and stealthy” liposomal formulation of the drug paclitaxel:

To prepare the drug liposome 65 mg of DSPC, 20 mg cholesterol, 10 mg DSPE-PEG, 5 mg DSPE-PEG maleimide and 10 mg paclitaxel dissolved in 1 ml ethanol are mixed until all components are in lipid solution. The lipid solution is dried under vacuum until a lipid film is formed. The lipid film is then hydrated using a 5 percent solution of human serum albumin. The hydrated lipid solution is then sonicated using a bath sonicator to prepare unilamella liposomes. The unilamella liposomes are then sized using a pressure extruder, by successive extrusions through membranes of decreasing pore sizes. Final extrusion through a membrane of 100 nm pore size results in the production of unilamella liposomes with a mean diameter of about 120 nm. The temperature of the constituents throughout this procedure is maintained at 60 C. which is above the transition temperature of the lipid mixture. The liposomes are stored at 4 C.

The unilamella liposomes thus formed are composed of a lipid bilayer membrane with the paclitaxel drug incorporated in the lipid layer. Liposomes thus prepared have anchored in the lipid layer the DSPE-PEG molecule with the distal end of the PEG chain being free. Also anchored in the lipid layer is the DSPE-PEG maleimide molecule with the distal end of the PEG chain bearing the active maleimide group being free. To attach the purified antinuclear antibody to the liposome the antibody molecule is first treated to prepare the Fab fragment which is chemically conjugated to the maleimide group. The Fab fragment is prepared by treating the antinuclear antibody molecule with an immobilized preparation of the enzyme papain in the presence of excess cysteine. The Fab fragments are then mixed with the liposomes where conjugation of the Fab fragment to the PEG-PE chain will occur through an amide linkage. The Fab bound liposomes thus formed are separated from unbound material by passage through a Sephadex column. The eluate fractions containing liposomes are tested using the ELISA test for ANA to confirm that the liposomes prepared in this manner have the active targeting antibody bound to their surface. The “smart and stealthy” drug liposomes thus prepared are stored at 4 C. until used. Alternatively, the drug liposomes are prepared with the DSPE-PEG maleimide component omitted from the lipid mixture. Instead the Fab antibody fragment is separately conjugated to the DSPE-PEG molecule and the conjugated compound is then incubated with the drug liposomes resulting in the insertion of the Fab DSPE-PEG compound into the lipid bilayer of the liposome.

In one embodiment of this invention the “smart and stealthy” nanoparticles or liposomes are prepared to have a mean diameter that will be within the range of 50 nm to 500 nm; preferably within the range of 50 nm to 400 nm; and more preferably within the range of 100 nm to 200 nm. It is well- known that many tumors have “leaky” capillaries with enlarged vascular pores and therefore selection of the drug nanoparticle or liposome to have a diameter between 100 nm and 200 nm will facilitate their penetration through the vascular bed (“vascular permeation effect”) and into the extracellular fluid space of the tumor tissue where they will accumulate. The antinuclear antibody attached to their surface will serve to anchor the drug nanoparticle or liposome within the disease site where the drug will be released for optimum effect.

Many infectious diseases and immune disorders also have “leaky” capillaries at the disease site. Therefore the “smart and stealthy” drug nanoparticles and drug liposomes designed for these diseases will likewise accumulate at the disease site where the drug will be released for optimum effect.

The above example of a “stealthy and smart” liposome is given by way of illustration and not of limitation. It is obvious to one of skill in the art that there are many different formulations of nanoparticles and liposomes that can be similarly developed, and to which the antinuclear antibody of this invention can be attached. It is also obvious to one of skill in the art that the targeting moiety of this invention can be the whole antibody molecule or the binding fragments F(ab)₂ and Fab of the antibody molecule. It is also obvious to one of skill in the art that a wide variety of pharmaceuticals can be incorporated into said nanoparticles and liposomes in order to carry the drug to the site of disease. These embodiments do not affect the novelty of this invention which is the use of autoimmune antinuclear antibodies as the targeting moiety of these “smart and stealthy” nanoparticles and liposomes; and they are therefore considered to be within the scope of this invention.

Pharmaceuticals that can be used to prepare drug nanoparticles or drug liposomes can be broadly classified into the following groups.

The cytotoxic drug group includes the folate inhibitors, pyrimidine analogs, purine analogs, alkylating agents and antibiotics. Specific examples include acivicin, aclarubicin, acodazole, adriamycin, ametantrone, aminoglutethimide, anthramycin, asparaginase, azacitidine, azetepa, bisantrene, bleomycin, busulfan, cactinomycin, calusterone, caracemide, carboplatin, carmustine, carubicin, chlorambucil, cisplatin, cyclophosphamide, cytarabine, dacarbazine, dactinomycin, daunorubicin, dezaguanine, diaziquone, doxorubicin, epipropidine, etoposide, etoprine, floxuridine, fludarabine, fluorouracil, fluorocitabine, hydroxyurea, iproplatin, leuprolide acetate, lomustine, mechlorethamine, megestrol acetate, melengestrol acetate, mercaptopurine, methotrexate, metoprine, mitocromin, mitogillin, mitomycin, mitosper, mitoxantrone, mycophenolic acid, nocodazole, nogalamycin, oxisuran, peliomycin, pentamustine, porfiromycin, prednimustine, procarbazine hydrochloride, puromycin, pyrazofurin, riboprine, semustine, sparsomycin, spirogermanium, spiromustine, spiroplatin, streptozocin, talisomycin, tegafur, teniposide, teroxirone, thiamiprine, thioguanine, tiazofurin, triciribine phosphate, triethylenemelamine, trimetrexate, uracil mustard, uredepa, vinblastine, vincristine, vindesine, vinepidine, vinrosidine, vinzolidine, zinostatin and zorubicin. Also included are the toxins such as ricin and diptheria toxin.

The angiogenesis inhibitors group includes: angiostatin, endostatin and tumstatin.

The antibiotic group includes: penicillin, cephalosporin, griseofulvin, bacitracin, polymyxin B, amphotericin B, erythromycin, neomycin, streptomycin, tetracycline, vancomycin, gentamicin, and rifamycin.

The anti-inflammatory group includes betamethasone, budesonide, cortisone, dexamethasone, hydrocortisone, methylprednisolone, prednisolone, prednisone, and triamcinolone.

The biological response modifier group includes cytokines such as tumor necrosis factor, interferons, angiostatin; immune depressors such as cyclosporine, sirolimus, and triptolide; and immune stimulators such as animal or microbial proteins.

This invention describes the therapeutic advantages of attaching a carrier antinuclear antibody to the surface of the drug nanoparticle or drug liposome in order to carry the pharmaceutical compound to the disease site. There are many methods of attaching an antinuclear antibody to the surface of the nanoparticle or liposome known to those skilled in the art. In some cases the antinuclear antibody is directly linked to the surface of the nanoparticle or liposome. In other cases the antinuclear antibody is attached through an intermediate link. For example, the antinuclear antibody is chemically linked to a polyethylene glycol (PEG) molecule that is itself bound to the surface of the nanoparticle or liposome.

There are many ways of preparing nanoparticles and liposomes; and there are many ways of attaching antinuclear antibodies to the surface of these structures. These procedures are known to those skilled in the art and do not affect the novelty of this invention, which is the unique use of antinuclear antibodies directed at intracellular elements to prepare “smart and stealthy” nanoparticles and “smart and stealthy” liposomes to treat cancer and other diseases.

The novelty of this invention to utilize antinuclear antibodies that are directed against intracellular components that are released into the extracellular environment means that the antinuclear antibody:pharmaceutical combinations derived from this invention are not tissue-specific but are in fact “necrosis-specific”. As many tumors and other diseases have damaged tissue with necrotic areas then these novel pharmaceuticals will have a very broad therapeutic profile and can be used to treat a wide variety of different diseases. For example the antinuclear antibody can be combined with a nanoparticle or liposomal formulation of a cytotoxic drug such as paclitaxel and used to treat a variety of tumors by causing the drug to localize within the areas of necrosis in the tumor; the same antinuclear antibody can be combined with a nanoparticle or liposomal formulation of an antibiotic such as penicillin and used to treat infectious disease by causing the antibiotic to localize within the areas of necrosis caused by the infective agent; and the same antinuclear antibody can be combined with a nanoparticle or liposomal formulation of an immune modulating drug such as cyclosporine and used to treat immune disorders such as rheumatoid arthritis by causing the drug to localize within the area of inflammation. It is obvious to one of skill in the art that a wide variety of pharmaceuticals can be combined with the antinuclear antibody of this invention to treat a wide variety of diseases.

In one embodiment of this invention the same antinuclear antibody can be attached to a variety of different drugs and used to treat a particular disease such as cancer: For example, the antinuclear antibody can be used to coat paclitaxel liposomes; the same antinuclear antibody can be used to coat methotrexate liposomes; and the same antinuclear antibody can be used to coat doxorubicin liposomes. It is obvious to one of skill in the art that there are a wide variety of cancer drugs that can be utilized to prepare a wide variety of targeting antibody:cancer drug formulations; and that the different targeted drug formulations can be employed singly or in combination to treat different types of cancer.

In one embodiment of this invention the same antinuclear antibody can be attached to a variety of antimicrobial drugs and used to treat an infection. For example, the antinuclear antibody can be used to coat penicillin liposomes; the same antinuclear antibody can be used to coat vancomycin liposomes; and the same antinuclear antibody can be used to coat erythromycin liposomes. It is obvious to one of skill in the art that there are a wide variety of antibiotics that can be utilized to prepare a wide variety of targeting antibody:antibiotic drug formulations; and that the different targeted drug formulations can be employed singly or in combination to treat different types of infection.

In one embodiment of this invention the same antinuclear antibody can be attached to a variety of anti-inflammatory and/or immune modulating drugs and used to treat an autoimmune disease such as arthritis. Many patients with rheumatoid arthritis or osteoarthritis have joint inflammation and damage occurring to the synovium. The antinuclear antibody can be used to coat prednisone liposomes; the same antinuclear antibody can be used to coat methotrexate liposomes; and the same antinuclear antibody can be used to coat cyclosporine liposomes. It is obvious to one of skill in the art that there are a wide variety of anti-inflammatory and immune modulating drugs that can be utilized to prepare a wide variety of targeting antibody:anti-inflammatory drug formulations; and that the different targeted drug formulations can be employed singly or in combination to treat different types of autoimmune diseases and inflammatory disorders.

Further, in one embodiment of this invention, because there are multiple different intracellular substances that are expressed in necrotic areas multiple different antinuclear antibody:drug combinations can be developed to treat a specific disease condition. For example, there would be a list of antinuclear antibodies developed against the various nuclear antigens as enumerated earlier. Each of these antinuclear antibodies can be attached to any one of the pharmaceutical drugs listed earlier to form a novel pharmaceutical. These novel drugs can then be used singly or in combination to treat a wide variety of diseases.

The novelty of this invention in treating cancer is the use of autoimmune antinuclear antibodies that are not targeted to tumor-associated antigens. For example, the carrier antinuclear antibodies of this invention that are utilized in treating tumors do not bind directly to viable tumor cells. Instead, the antinuclear antibodies are directed against normal intracellular components present in both tumor and normal cells that are expressed into the extracellular medium in the areas of necrosis. As such the antinuclear antibodies have no direct influence upon the tumor cells. However, they are utilized to carry the therapeutic drug to necrotic areas within the tumor where the drug can be released and affect the surrounding tumor cells. There are no prior reports of the use of nanoparticles and/or liposomes labeled with autoimmune antinuclear antibodies being employed in cancer treatment.

Similarly, the novelty of this invention in treating infectious disease is the use of autoimmune antinuclear antibodies that are not targeted to the disease causing organism. Instead, the antinuclear antibodies are directed against normal intracellular components that are expressed into the extracellular medium in the areas of necrosis. As such the antinuclear antibodies have no direct influence upon the disease causing organism. However, they are utilized to carry the therapeutic drug to the site of infection where the drug can be released and affect the pathogen. There are no prior reports of the use of nanoparticles or liposomes labeled with autoimmune antinuclear antibodies being employed in the treatment of infectious disease.

Similarly, the novelty of this invention in treating immune disorders is the use of autoimmune antinuclear antibodies that are not targeted to the systemic immune system. Instead, the antinuclear antibodies are directed against normal intracellular components that are expressed into the extracellular medium in the areas of inflammation. As such the antinuclear antibodies have no direct influence upon the inflammatory cells. However, they are utilized to carry the therapeutic drug to the inflamed site where the drug can be released and affect the inflammatory cells within the area of inflammation. There are no prior reports of the use of nanoparticles or liposomes labeled with autoimmune antinuclear antibodies being employed in the treatment of immune disorders.

Depending on the disease to be treated patents will generally receive these drugs parenterally, or by intramuscular injection or by subcutaneous injection, or by direct injection into the disease site. Being human derived, the carrier antinuclear antibodies are non-immunogenic and patients can therefore receive repeated treatment without developing an allergic reaction to the antinuclear antibody.

The description and examples presented in this invention are gives as illustration and not as limitation. Those of skill in the art will recognize from the description and examples given in this invention other variations, embodiments and applications that fall within the concept, spirit and scope of this invention.

What is claimed is:

1. A method of utilizing autoimmune antinuclear antibodies as a targeting agent for drug nanoparticles and/or drug liposomes used in the treatment of cancer and other diseases.

2. A method according to claim 1 whereby the autoimmune antinuclear antibodies have the capacity to bind to intracellular components of the cell that are expressed extracellularly within necrotic areas found in tumors, and/or in infectious diseases, and/or in autoimmune diseases and/or at sites of inflammation.

3. A method according to claim 2 whereby the intracellular components being targeted by the antinuclear antibody is nuclear material that is released from dead cells into the extracellular environment.

4. A method according to claim 1 whereby the autoimmune antinuclear antibodies are obtained from patients with autoimmune disease either by blood donation, and/or by apheresis, and/or using human hybridomas, and/or transgenic animals.

5. A method according to claim 1 whereby the antinuclear antibodies are purified by an affinity binding method.

6. A method whereby the antinuclear antibodies are attached to the surface of drug nanoparticles through a polyethylene glycol molecule link.

7. A method whereby the antinuclear antibodies are attached to the surface of drug liposomes through a polyethylene glycol molecule link.

8. A method according to claim 6 whereby the drug nanoparticles are prepared from a drug that is insoluble or poorly soluble in physiological solution.

9. A method according to claim 7 whereby the drug liposomes are prepared from a drug that is lipid soluble but insoluble or poorly soluble in physiological solution

10. A method according to claim 7 whereby the drug liposomes are prepared from a drug that is soluble in aqueous or physiological solution.

11. A method according to claim 1 of cancer treatment utilizing a therapeutic dosage of an anti-cancer drug formulated as antinuclear antibody coated nanoparticles or antinuclear antibody coated liposomes and injected into the patient.

12. A method according to claim 1 of cancer treatment utilizing a therapeutic dosage of a biological response modifier formulated as antinuclear antibody coated nanoparticles or antinuclear antibody coated liposomes and injected into the patient.

13. A method according to claim 1 of cancer treatment utilizing a therapeutic dosage of a toxin formulated as antinuclear antibody coated nanoparticles or antinuclear antibody coated liposomes and injected into the patient

14. A method according to claim 1 of cancer treatment utilizing a therapeutic dosage of a foreign animal or microbial protein formulated as antinuclear antibody coated nanoparticles or antinuclear antibody coated liposomes and injected into the patient.

15. A method according to claim 1 of cancer treatment utilizing a therapeutic dosage of an angiogenesis inhibiting compound formulated as antinuclear antibody coated nanoparticles or antinuclear antibody coated liposomes and injected into the patient.

16. A method according to claim 1 for treatment of infectious disease utilizing a therapeutic dosage of an antibiotic or anti-microbial drug formulated as antinuclear antibody coated nanoparticles or antinuclear antibody coated liposomes and injected into the patient

17. A method according to claim 1 for treatment of immune diseases and inflammatory disorders such as rheumatoid arthritis and osteoarthritis utilizing a therapeutic dosage of a steroidal, or cytotoxic, or immune modulating drug formulated as antinuclear antibody coated nanoparticles or antinuclear antibody coated liposomes and injected into the patient.
